(19) United States
(12) Patent Application Publication
(10) Pub. No.: US 2008/0033577 A1
(43) Pub. Date: Feb. 7, 2008

(54) Hip Resurfacing Component

(76) Inventor: Lawrence Kohan, Bondi Junction (AU)

Correspondence Address: MCCARTER & ENGLISH , LLP STAMFORD OFFICE, FINANCIAL CENTRE , SUITE 304A, 695 EAST MAIN STREET, STAMFORD, CT, 06901-2138, US

(21) Appl. No.: 11/630,546

(22) PCT Filed: Jul. 1, 2005
(86) PCT No.: PCT/AU05/00970

§ 371 Date: Jun. 27, 2007

(30) Foreign Application Priority Data
Jul. 1, 2004 (AU) 2004903623

(51) Int. Cl. A61F 2/60; A61B 17/00

(52) U.S. Cl. 623/32; 606/79; 606/84

(57) ABSTRACT

A femoral component (1) for a hip resurfacing arthroplasty comprising a femoral cap (2), adapted to engage with a cup (3) which is set into a pelvic bone (15), the femoral cap having a convex (4) surface and a concave surface (5), and a stem (6) attached to the concave surface of the femoral cap, characterised in that at least a portion of the stem is composed of a soluble material.

### TECHNICAL FIELD

The present invention relates to hip resurfacing, and in particular to a femoral component for a hip resurfacing arthroplasty. However, the invention is not limited to hip resurfacing and can be utilised for other ball and socket joint applications in humans and other mammals.

### BACKGROUND

The concept of hip resurfacing has been known since the late 1950s. Hip resurfacing relies on the fact that it is preferable to replace only the bone surfaces within a weakened or diseased hip rather than radically removing large portions of bone. This approach has the benefit of preserving the femoral head and neck. This leaves the natural off-set and anteversion of the hip joint intact and maintains approximate leg length equivalence. The larger size of the ball in the ball and socket joint diminishes the problem of dislocation. The stress loading on the bone is relatively natural. Further, metals, which have a low wear rate can be used.

In a hip resurfacing arthroplasty the diseased portion of the pelvic socket is removed. A replacement cup is set into the pelvic bone. The articular surfaces of the femur and the femur head are reshaped and a femoral cap is set onto the femur and adapted to engage with the cup.

In order to assist in aligning the cap with the femur during surgery, the cap includes a stem. The stem allows for alignment of the cap and also stability of the joint until the bone meshes with the metal and/or cement of the femoral cap.

This stem, while beneficial, can produce weakening of the femur, along with microfracture and unnatural stress within the femur bone. This can cause significant pain for a patient and, long term, may weaken the femur.

Attempts have been made to make the stem removable in order to minimise the trauma suffered by the patient and the consequently relatively long recovery period and subsequent hospital stay. One such device is described in GB 2372707 (McMinn). In that disclosure the stem comprises a first stem portion connected to the femoral cap and a second removable stem portion. Whilst in this device there is the advantage of removing the second stem portion, the remaining portion remains in the femoral head. Some similar disadvantages to the earlier prior art exist with the remaining first stem portion.

It is an object of the present invention to provide a femoral component for a hip resurfacing arthroplasty which will overcome or ameliorate at least some of the deficiencies in the prior art, or to at least provide an alternative.

It is a further object of the present invention to provide a bone component for a ball and socket resurfacing arthroplasty for a mammal, including a human, which will overcome or at least ameliorate at least some of the deficiencies in the prior art, or to at least provide an alternative.

### SUMMARY OF THE INVENTION

According to a first aspect the present invention consists of a femoral component for a hip resurfacing arthroplasty comprising a femoral cap, adapted to engage with a cup which is set into a pelvic bone, the femoral cap having a convex surface and a concave surface, and a stem attached to the concave surface of the femoral cap, characterised in that at least a portion of the stem is composed of a soluble material.

Preferably, in a first embodiment the concave surface of the femoral cap includes a stem cavity.

Preferably, the stem is wholly composed of a soluble material.

Preferably, in a particular embodiment, the stem is composed of a magnesium alloy.

Preferably, in an alternative embodiment, the stem is composed of an aluminium or zinc alloy.

Preferably, in a further alternative embodiment, the stem is composed of an acid, such as polyglycolic acid or polylactic acid.

Preferably, in a further alternative embodiment, the stem is composed of a degradable polymer, such as trimethylene carbonate copolymer.

Preferably, in a further alternative embodiment, the stem is composed of hydroxy apatite.

Preferably, the soluble material assists in osteogenesis.

Preferably, in one embodiment the stem cavity and the upper end of the stem are threaded.

Preferably, in a second embodiment, the stem cavity and the upper end of the stem include a bayonet fitting.

Preferably, in a third embodiment, the stem cavity and the upper end of the stem include a truncated cone fitting.

Preferably in a fourth embodiment, the stem cavity and the upper end of the stem include a trunnion fitting.

According to a second aspect the present invention consists of a femoral component for a hip resurfacing arthroplasty comprising a femoral cap, adapted to engage with a cup which is set into a pelvic bone, the femoral cap having a convex surface and a concave surface, and a stem attached to the concave surface of the femoral cap, characterised in that at least a portion of the stem is capable of being dissolved.

According to a third aspect the present invention consists of a bone component for a ball and socket joint resurfacing arthroplasty in a mammal, including a human, comprising a stem characterised in that at least a portion of the stem is composed of a soluble material.

Preferably, in a first embodiment, the ball and socket joint is a hip joint.

Preferably, in a second embodiment, the ball and socket joint is a shoulder joint.

Preferably, in one embodiment, the stem is composed of a magnesium alloy.

Preferably, in an alternative embodiment the stem is composed of an aluminium or zinc alloy.

Preferably, in an alternative embodiment the stem is composed of a polyglycolic acid.

According to a fourth aspect of the present invention, described is a bone component for a ball and socket joint arthroplasty in a mammal, including a human, comprising a stem characterised in that at least a portion of said stem is capable of being dissolved.

### DESCRIPTION OF THE FIGURES

A preferred embodiment of the invention will now be described by way of example only, with reference to the accompanying figures in which:

FIG. 1 is a cross sectional view of a femoral component for a hip resurfacing arthroplasty in accordance with a third preferred embodiment.

FIG. 2 is a cross sectional view of the femoral component of FIG. 1.

FIG. 3 is a perspective view of the femoral component of FIG. 1.

### BEST MODE OF THE INVENTION

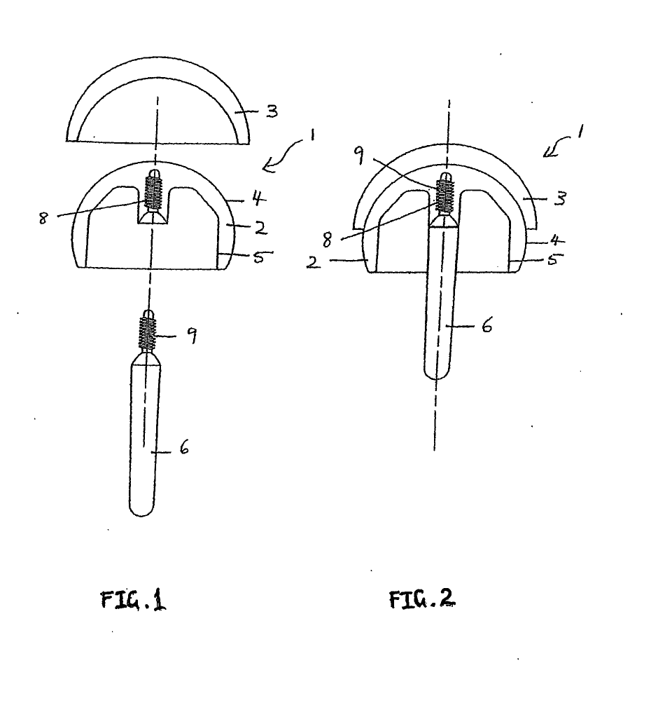

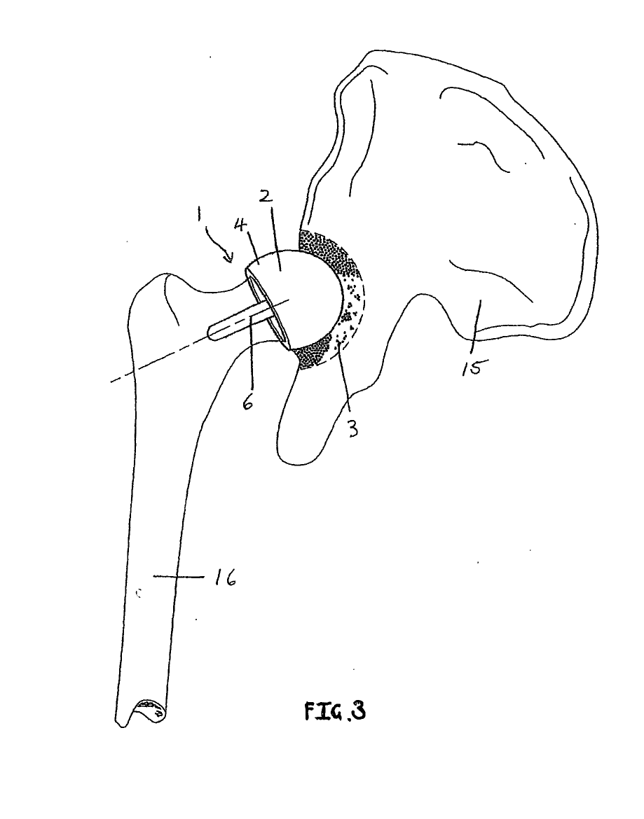

Referring to FIGS. 1-3 there is shown a femoral component 1 for a human hip resurfacing arthroplasty. The femoral component 1 comprises a femoral cap 2 with a convex surface 4 and a concave surface 5. The femoral cap 2 is adapted to engage with a cup 3. The femoral cap 2 and cup 3 are composed of a metal, such as steel.

The femoral cap 2 is adapted to be set onto a femoral bone 16 and also adapted to engage with the cup 3 that is set into the pelvic bone 15 of the patient. It can be seen that in use this engagement of the femoral cap 2 and cup 3 comprises a ball and socket joint.

The femoral component 1 further comprises a stem 6 that is adapted to be attached to femoral cap 2. The stem 6 is adapted to be removable from the femoral cap 2.

The femoral cap 2 includes threaded female cavity 8. The stem 6 has an upper end threaded end 9, which is adapted to engage with the female cavity 8. It can be seen that this allows the stem 6 to be readily removed from the femoral cap 2.

Stem 6 is composed of “soluble” material, such as soluble metal, a degradable polymer or an acid. The soluble metal may be a magnesium alloy, an aluminium zinc alloy or any other soluble metal alloy. The degradable polymer may be trimethylene carbonate copolymer. The acid may be polyglycolic or polylactic acid. Another soluble material that may be used is hydroxy apatite. It should also be understood that a combination of these or other absorbable materials, could be used.

By “soluble” we mean materials that are capable of being dissolved or liquefied. Suitable materials must be sufficiently rigid when inserted into the body to allow for easy insertion but will dissolve over time.

An advantage of using a magnesium alloy is that it may assist in osteogenesis and the resultant stimulus in bone growth. This helps to ensure that the bone regrowth that occurs in femur 16 happens relatively quickly. Therefore, the recovery time and length of the hospital stay of the patient may be reduced.

In this embodiment, the stem 6 is preferably removed in the process of dissolving as a result of the influence of the patient's body's natural processes acting on the soluble material of the stem 6.

It can be seen that upon placement, the soluble stem 6 will dissolve over time and femur 16 will regrow to fill the space left when stem 6 dissolves. The advantage of removing stem 6 from the femoral bone 16 is that the bone is subjected to natural rather than shielded stress as it heals. This has the advantage that as the bone heals, there is less chance for the bone to fracture or break.

In use a guide wire (not illustrated) is inserted through the femur 16 from the lateral side. A cannulated drill (not illustrated) is utilised to drill a channel (not illustrated) from the lateral side of the femur 16 through the femur head. The femoral component 1 is fitted to the femur head and the stem 6 forces the cannulated drill from the drilled channel. Stem 6 is then removed from the channel and the channel is packed with bone graft to stimulate bone regrowth.

The foregoing describes only preferred embodiments of the present invention and modification, obvious to those skilled in the art, can be made thereto without departing from the scope of the present invention.

It is envisaged that the engagement between the stem 6 and the femoral cap 2 could comprise any type of fitting including a bayonet fitting, a truncated cone fitting and trunnion fitting.

The femoral cap 2 and cup 3 are composed of steel, however, any other appropriate material, such as cobalt chrome, may be used.

Whilst the femoral component has been described for use in hip surfacing arthroplasty, the present invention could be used for any ball and socket joint replacement in humans or other mammals.

Whilst in the abovementioned embodiment the stem 6 is completely soluble, it should be understood that in other not shown embodiments, any portion of stem 6 may be composed of a soluble material. For example, the upper threaded end 9 of stem 6 may be composed of a metal such as steel while the remaining portion may be composed of a soluble material.

In addition to the body's natural processes acting on the soluble material of stem 6, in order to help speed up the process of dissolving stem 6, suitable catalysts such as pharmaceuticals or other medical treatments administered to the patient may be applied.

The term “comprising” as used herein is used in the inclusive sense of “having” or “including”, and not in the exclusive sense of “consisting only of”.

1. A femoral component for a hip resurfacing arthroplasty comprising:
a femoral cap, adapted to engage with a cup which is set into a pelvic bone, the femoral cap having a convex surface and a concave surface, and
a stem attached to the concave surface of the femoral cap, characterised in that at least a portion of said stem is composed of a soluble material.

2. A femoral component for a hip resurfacing arthroplasty as claimed in claim 1, wherein the concave surface of the femoral cap includes a stem cavity.

3. A femoral component for a hip resurfacing arthroplasty as claimed in claim 1, wherein the stem is wholly composed of a soluble material.

4. A femoral component for a hip resurfacing arthroplasty as claimed in any of the preceding claims, wherein the stem is composed of a magnesium alloy.

5. A femoral component for a hip resurfacing arthroplasty as claimed in any of claims 1 to 3, wherein the stem is composed of an aluminium or zinc alloy.

6. A femoral component for a hip resurfacing arthroplasty as claimed in any of claims 1 to 3, wherein the stem is composed of an acid, such as polyglycolic acid or polylactic acid.

7. A femoral component for a hip resurfacing arthroplasty as claimed in any of any of claims 1 to 3, wherein the stem is composed of a degradable polymer, such as trimethylene carbonate copolymer.

8. A femoral component for a hip resurfacing arthroplasty as claimed in any of any of claims 1 to 3, wherein the stem is composed of hydroxy apatite

9. A femoral component for a hip resurfacing arthroplasty as claimed in claim 1, wherein the soluble material assists in osteogenesis.

10. A femoral component for a hip resurfacing arthroplasty as claimed in claim 2, wherein the stem cavity and the upper end of the stem are threaded.

11. A femoral component for a hip resurfacing arthroplasty as claimed in claim 2, wherein the stem cavity and the upper end of the stem include a bayonet fitting.

12. A femoral component for a hip resurfacing arthroplasty as claimed in claim 2, wherein the stem cavity and the upper end of the stem include a truncated cone fitting.

13. A femoral component for a hip resurfacing arthroplasty as claimed in claim 2, wherein the stem cavity and the upper end of the stem include a trunnion fitting.

14. A femoral component for a hip resurfacing arthroplasty comprising:
a femoral cap, adapted to engage with a cup which is set into a pelvic bone, the femoral cap having a convex surface and a concave surface, and
a stem attached to the concave surface of the femoral cap, characterised in that at least a portion of said stem is capable of being dissolved.

15. A bone component for a ball and socket joint resurfacing arthroplasty in a mammal, including a human, comprising a stem characterised in that at least a portion of said stem is composed of a soluble material.

16. A bone component for a ball and socket joint resurfacing arthroplasty as claimed in claim 14, wherein said ball and socket joint is a hip joint.

17. A bone component for a ball and socket joint resurfacing arthroplasty as claimed in claim 14, wherein said ball and socket joint is a shoulder joint.

18. A bone component for a ball and socket joint arthroplasty as claimed in claim 14, wherein the stem is composed of a magnesium alloy.

19. A bone component for a ball and socket joint arthroplasty as claimed in claim 14, wherein the stem is composed of a aluminium or zinc alloy.

20. A bone component for a ball and socket joint arthroplasty as claimed in claim 14, wherein the stem is composed of a degradable polymer, such as trimethylene carbonate copolymer.

21. A bone component for a ball and socket joint arthroplasty as claimed in claim 14, wherein the stem is composed of an acid, such as polyglycolic acid or polylactic acid.

22. A bone component for a ball and socket joint arthroplasty in a mammal, including a human, comprising a stem characterised in that at least a portion of said stem is capable of being dissolved.
